(19) United States
(12) Patent Application Publication
(10) Pub. No.: US 2016/0199621 A1
(43) Pub. Date: Jul. 14, 2016

(54) MAGNETICALLY INSERTABLE WIRE MATERIALS

(71) Applicant: FORT WAYNE METALS RESEARCH PRODUCTS CORP, Fort Wayne, IN (US)

(72) Inventor: Jeremy E. SCHAFFER, Leo, IN (US)

(21) Appl. No.: 14/911,717

(22) PCT Filed: Aug. 13, 2014
(86) PCT No.: PCT/US14/50933

§ 371 Date: Feb. 11, 2016

Related U.S. Application Data
(60) Provisional application No. 61/865,852, filed on Aug. 14, 2013.
(60) Provisional application No. 61/907,438, filed on Nov. 22, 2013.

(51) Int. Cl. A61M 25/09; H01B 5/02; A61M 25/01; H01B 1/02

(52) U.S. Cl. CPC A61M 25/09; H01B 1/02; H01B 5/02; A61M 25/0127; A61M 2025/09091

(57) ABSTRACT

A wire construct is ferromagnetic and/or biocompatible, while also having a size and geometry appropriate for use as an electrode for insertion into an in vivo implant site. In ferromagnetic embodiments, the wire is magnetically insertable via an electromagnetic driver, which uses high-speed magnetic acceleration to drive the wire material into a neural or other implant site. A helical thread may be formed on the external surface of the wire to facilitate rotational fine positioning after initial implantation. The wire may be formed as a monolithic structure with a magnetic metal alloyed with an inert and biocompatible metal, or may be a bimetallic structure in which a magnetic outer shell has an inert and biocompatible inner core. The wire is sized to allow transmission of electrical signals either to or from the implant site while avoiding immune response, thereby ensuring a long service life.

### CROSS REFERENCE TO RELATED APPLICATIONS

This application claims the benefit under 35 U.S.C. §119(e) of U.S. Provisional Patent Application Ser. No. 61/865,852 filed Aug. 14, 2013 and U.S. Provisional Patent Application Ser. No. 61/907,438 filed Nov. 22, 2013, both entitled MAGNETICALLY INSERTABLE WIRE MATERIALS, the entire disclosures of which are hereby expressly incorporated herein by reference.

### BACKGROUND

1. Technical Field

The present invention relates to biodegradable wire used in biomedical applications and, in particular, relates to wires used as magnetically insertable electrodes.

2. Description of the Related Art

Electrodes, when inserted in the human brain, facilitate the flow of electronic signals to and from the brain. Specifically, these signals can permit neural recording and stimulation, which have great potential for clinical applications, such as treating neurological conditions like Parkinson's disease or epilepsy, or for controlling prosthetic limbs. However, long-term neural recording may be impaired by eventual failure of implantable electrodes, such as failure due to a tissue response caused by the implantation and constant presence of the electrode. In these cases, astrocytes and microglia (among other physical defenses in the brain) attempt to engulf the electrode, increasing the electrical impedance between the electrode and neurons, and possibly pushing neurons away from the recording site.

What is needed is an improvement over the foregoing.

### SUMMARY

A wire construct is ferromagnetic and/or biocompatible, while also having a size and geometry appropriate for use as an electrode for insertion into an in vivo implant site. In ferromagnetic embodiments, the wire is magnetically insertable via an electromagnetic driver, which uses high-speed magnetic acceleration to drive the wire material into a neural or other implant site. A helical thread may be formed on the external surface of the wire to facilitate rotational fine positioning after initial implantation. The wire may be formed as a monolithic structure with a magnetic metal alloyed with an inert and biocompatible metal, or may be a bimetallic structure in which a magnetic outer shell has an inert and biocompatible inner core. The wire is sized to allow transmission of electrical signals either to or from the implant site while avoiding immune response, thereby ensuring a long service life.

In one form thereof, the present disclosure provides an elongate, flexible wire having a maximum diameter between 5 μm and 200 μm and a composition including a combination of a ferromagnetic metal and a biocompatible metal.

In another form thereof, the present disclosure provides a bimetallic wire, made for insertion at an implant site, the wire including a shell made of a ferromagnetic metal a core disposed within the shell, the core made of a biocompatible metal, the core defining a core diameter between 5 μm and 25 μm.

In yet another form thereof, the present disclosure provides an elongate, flexible electrode wire for use at an implant site, the wire having a maximum diameter between 5 μm and 200 μm, a composition comprising a biocompatible metal and a helically-grooved outer surface.

### BRIEF DESCRIPTION OF THE DRAWINGS

The above mentioned and other features and objects of this invention, and the manner of attaining them, will become more apparent and the invention itself will be better understood by reference to the following description of embodiments of the invention taken in conjunction with the accompanying drawings, wherein:

FIG. 1 is an elevation, cross-sectional view of a monolithic wire of the present disclosure;

FIG. 2a is an elevation, cross-sectional view of a bimetallic wire of the present disclosure;

FIG. 2b is a perspective view of another bimetallic wire of the present disclosure, including a coating;

FIG. 3a is a schematic view illustrating an exemplary forming process of monolithic wire using a lubricated drawing die;

FIG. 3b is a schematic view illustrating an exemplary forming process of composite wire using a lubricated drawing die;

FIG. 4 is an elevation view of a wire in accordance with the present disclosure, before a final cold working process;

FIG. 5 is an elevation view of the wire of FIG. 4, after the final cold working process;

FIG. 6 is a perspective view of an apparatus for high-speed magnetic insertion of a wire into the skull of a laboratory animal;

FIG. 7a shows an elevation view of a monolithic wire made in accordance with the present disclosure and featuring a pointed tip;

FIG. 7b shows an elevation view of a bimetallic wire made in accordance with the present disclosure and featuring a pointed tip;

FIGS. 8a-8c show an elevation, cross-sectional view of progressively smaller cross-sections of a wire construct during an iterative drawing process in accordance with the present disclosure;

FIG. 8d shows an elevation, cross-sectional view of a wire in accordance with the present disclosure, resulting from the iterative drawing process of FIGS. 8a-8c;

FIG. 9a shows a perspective view of the wire of FIG. 8d;

FIG. 9b shows an elevation view of the wire of FIG. 8d positioned between two twisting grips;

FIG. 10a shows a perspective view of the wire of FIG. 8d after having undergone twisting between the two grips shown in FIG. 9b;

FIG. 10b shows a perspective view of the wire of FIG. 8d being twisted between the two grips shown in FIG. 9b;

FIG. 11a shows a perspective view of a wire construct in accordance with alternative embodiment of the present disclosure;

FIGS. 11b-11d show perspective views illustrating the wire construct of FIG. 11a as part of a drawing assembly, in which the drawing assembly has progressively smaller cross-sections during an iterative drawing process in accordance with the present disclosure; and

FIG. 11e shows a perspective view of a wire in accordance with the present disclosure, resulting from the iterative drawing process of FIGS. 11a-11d and removed from the drawing assembly.

Corresponding reference characters indicate corresponding parts throughout the several views. Although the exemplifications set out herein illustrate embodiments of the invention, the embodiments disclosed below are not intended to be exhaustive or to be construed as limiting the scope of the invention to the precise form disclosed.

### DETAILED DESCRIPTION

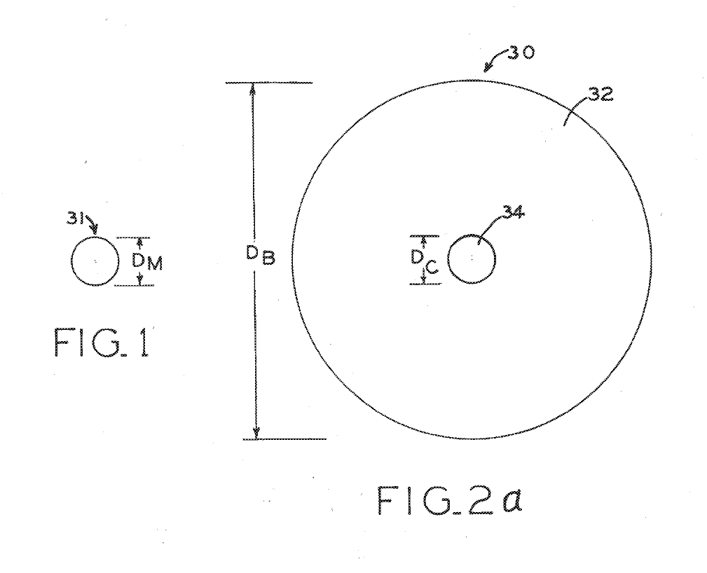

Material made in accordance with the present disclosure may be formed into wire for use as an electrode that is magnetically inserted at a high speed into the brain or other organ or implant site. In one embodiment, monolithic wire 31 (FIG. 1) is made of a ferromagnetic electrode alloy described in further detail below. Monolithic wire 31 may be formed with a uniform size and cross-sectional geometry along its axial length, such as with a round cross-sectional shape having outer diameter D_{M} as shown in FIG. 1. In an exemplary embodiment, diameter D_{M} may range between approximately 12 and 25 microns.

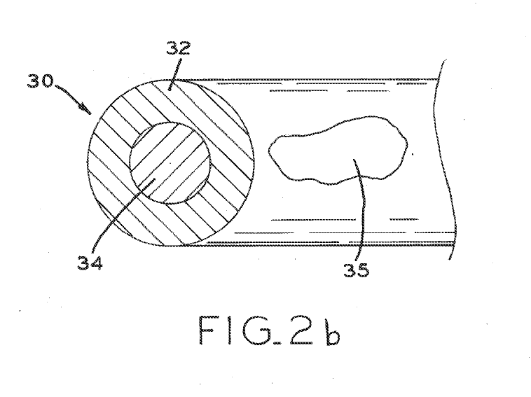

In another embodiment, bimetallic wire 30 (FIG. 2a) may include a separate core 34 and shell 32, in which core 34 functions as an electrode material and shell 32 provides a temporary, bioabsorbable casing to facilitate implantation of wire 30 at a desired depth. In an exemplary embodiment, bimetallic wire 30 has a total outer diameter D_{B} which may be as large as approximately 200 microns.

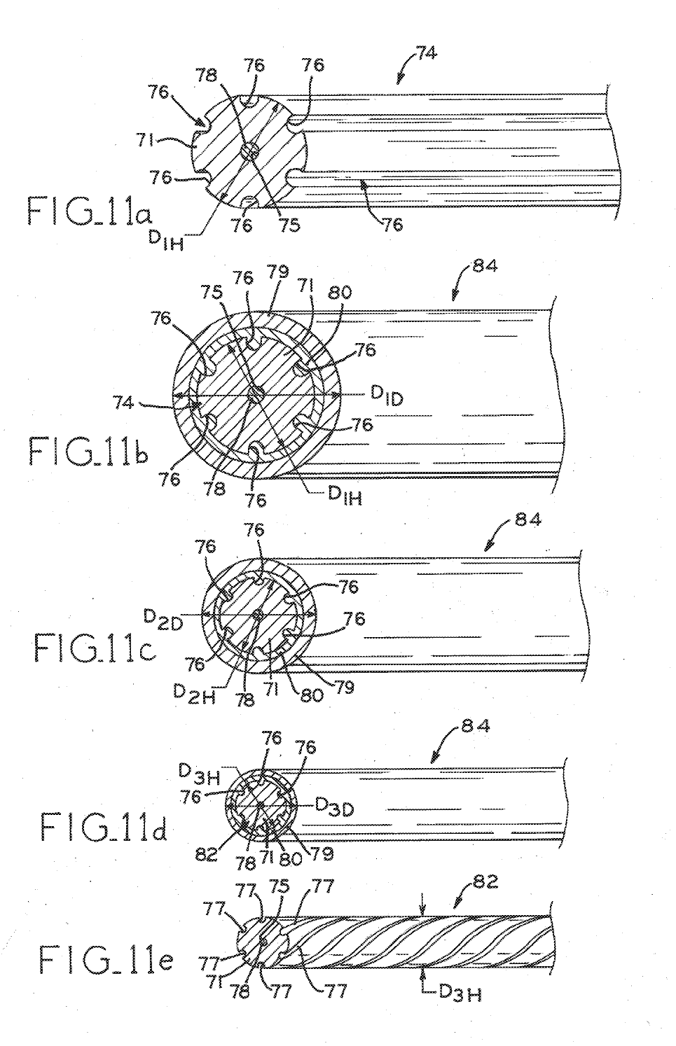

Monolithic wire 31 and bimetallic wire 30 may be formed into threaded wires 68 or 82 (FIGS. 10a and 11 e) which respectively include a helical threadform 72, 77 on its outer longitudinal surface. As described in further detail below, threadform 72, 77 may engage the surrounding tissue (e.g., bone) after initial implantation in order to facilitate fine axial adjustment at the implant site by rotating wires 68 or 82.

As described in further detail below, outer diameters D_{M}, D_{B}, D_{3H} of wires 31, 30, 68/82 may be respectively tailored to balance competing interests in the context of a magnetic electrode insertion and subsequent in vivo use. On one hand, faster insertion speed, finer tip geometry, smaller size, and lower material stiffness has been found to decrease damage caused by the insertion process, and reduces the intensity of the reactive tissue response. On the other hand, small electrode wires may buckle during insertion as they encounter bodily tissue, particularly for rigid tissues (e.g., bone) and/or deeper implant sites. Wire made in accordance with the present disclosure may be sized, shaped and constructed to provide strength sufficient to resist or avoid such buckling, while also minimizing the intensity of the reactive tissue response, and offering a consistently low impedance over a long service life.

### TERMINOLOGY

As used herein, “biodegradable,” “bioabsorbable” and “bioresorbable” all refer to a material that is able to be chemically broken down in a physiological environment, i.e., within the body or inside body tissue, such as first by corrosion, then by biological processes including resorption and absorption. This process of chemical breakdown will generally result in the complete structural degradation of the material and/or appliance within a period of weeks to months, such as 18 months or less, 24 months or less, or 36 months or less, for example. This rate stands in contrast to more “degradation-resistant” or permanent materials and/or appliances, such as those constructed from nickel-titanium alloys (“Ni—Ti”), stainless steel or inert precious metals, for example. These degradation-resistant materials remain in the body, structurally intact, for a period exceeding at least 36 months and potentially throughout the lifespan of the recipient. Biodegradable metals used herein include nutrient metals, i.e., metals such as iron, magnesium, manganese and alloys thereof. These nutrient metals and metal alloys have biological utility in mammalian bodies and are used by, or taken up in, biological pathways.

As used herein, “magnetic” materials are metals, such as iron, nickel, or cobalt, in which material atoms have one or more unpaired electrons resulting in a net magnetic moment in the absence of an external magnetic field. When the spin of these unpaired electrons line up parallel with each other in a given area (the area known as the magnetic or ferromagnetic domain), there is an independent magnetic moment created in that domain. Thus, magnetic materials are those materials that upon which an attractive or repulsive force is exerted in the presence of an external magnetic field acting upon the magnetic material. Exemplary magnetic materials include iron, steel, and alloys thereof.

As used herein, “magnesium ZM21” refers to magnesium ZM21 alloy, otherwise known as ZM-21 or simply ZM21 alloy, which is a medium-strength forged magnesium alloy comprising 2 wt % Zn, 1 wt % Mn and a balance of Mg.

“Fe(II)” refers to iron ions of charge 2+ that may be associated with degradation products in a saline or bodily environment of iron or iron based alloys.

“Fe(III)” refers to iron ions of charge 3+ that may be associated with degradation products in a saline or bodily environment of Fe or Fe-based alloys.

“Mg(II)” refers to magnesium ions of charge 2+ that may be associated with degradation products in a saline or bodily environment of Mg or Mg-based alloys.

“RE” is used here to signify the rare earth elements given in the periodic table of elements and including elements such as Scandium, Yttrium, and the fifteen lanthanides, i.e. La, Ce, Pr, Nd, Pm, Sm, Eu, Gd, Tb, Dy, . . . , to Lu.

“DFT®” is a registered trademark of Fort Wayne Metals Research Products Corp. of Fort Wayne, Ind., and refers to a bimetal or poly-metal composite wire product including two or more concentric layers of metals or alloys, typically at least one outer layer disposed over a core filament formed by drawing a tube or multiple tube layers over a solid metallic wire core element.

“OD” refers to the outside diameter of a metallic wire or outer shell.

“ID” refers to the inside diameter of a metallic outer shell.

### Monolithic Wire

1. Material Characteristics

Referring to FIG. 1, a cross-section of monolithic wire 31 (constructed of a single alloy) is shown. Wire 31 is an elongated, flexible and generally cylindrical construct having diameter D_{M} in cross-section, as shown.

In one exemplary embodiment, wire 31 is formed of a Pt—Fe alloy, such as an alloy having 40 wt. % Fe and balance Pt. Fe renders the alloy ferromagnetic and thus, compatible with a high-speed magnetic insertion process into an in vivo implant site, which will be described in greater detail below. On the other hand, Pt renders the alloy corrosion-resistant to produce a biocompatible material capable of conveying electrical signals, such that wire 31 is useable as an implantable electrode for, e.g., transmitting electrical impulses or signals to or from a brain. The relative proportions of Fe and Pt in wire 31 may vary, with materials in accordance with the present disclosure having Fe in an amount as little as 20 wt. %, 30 wt. % or 40 wt. % and as much as 50 wt. %, 60 wt. % or 70 wt. %, or may include any amount of Fe within any range defined by any two of the foregoing values.

The present Pt—Fe alloys may be sized, shaped and processed to have a desired strength, such as for magnetic insertion at a particular implant site. For example, wire 31 may be cold-worked into a final form (by, e.g., drawing of the wire as described below) or otherwise wrought or mechanically conditioned to enhance the elasticity, ultimate tensile strength and/or yield strength of the material. Such enhancement of mechanical properties of the wire materials allows the wire to be tailored for use at a wider range of in vivo sites, such as in extremities where more extreme wire bends can be expected.

2. Geometric Characteristics

Referring again to FIG. 1, monolithic wire 31, having diameter D_{M}, is sized and shaped to have sufficient strength to facilitate penetration to a desired depth at an implantation site, without buckling. At the same time, diameter D_{M} is small enough to avoid triggering the human body's natural defenses which might otherwise promote degradation of wire 31 in situ.

With respect to buckling, Fe-alloy wires may encounter interference during high-speed magnetic insertion due to potential tissue impacts during penetration into the human brain (or other body part) that operate to urge the wire to bend or warp. Diameter D_{M} of wire 31 is sized small enough to minimize interference during travel through body (e.g., brain) tissue and to maximize the delivery speed of the high-speed magnetic insertion system, while remaining large enough to provide the columnar strength needed for a particular implantation site (e.g., implant depth, tissue density, etc.). For neural applications of wire 31, such as an electrode inside a human or other mammalian brain, diameter D_{M} is also sized small enough to facilitate long-term implant functionality. For example, diameter D_{M} may be maintained at less than or equal to the diameter of a human hair, such that glial cells (such as strocytes and microglia, which are parts of the human brain's physical defences) are less prone to attack the electrode. In one exemplary embodiment, wire 31 balances all of the above competing interests by having a diameter D_{M} as small as 5 μm, 9 μm or 12 μm, and as large as 15 μm, 20 μm, or 25 μm, or any diameter within any range defined by any of the foregoing values.

Although a wire having a round cross-section is shown in FIG. 1, non-round wire forms may also be produced using the materials disclosed herein. Other exemplary forms include polygonal cross-sectional shapes such as rectangular cross-sectional shapes and hollow forms such as tubing, which may be used directly in an end product or as a shell in bimetallic composite wire 30 (further described below). For purposes of the present disclosure, the “diameter” of a wire form non-round cross-sectional shape is the smallest circle circumscribing the non-round cross-sectional shape.

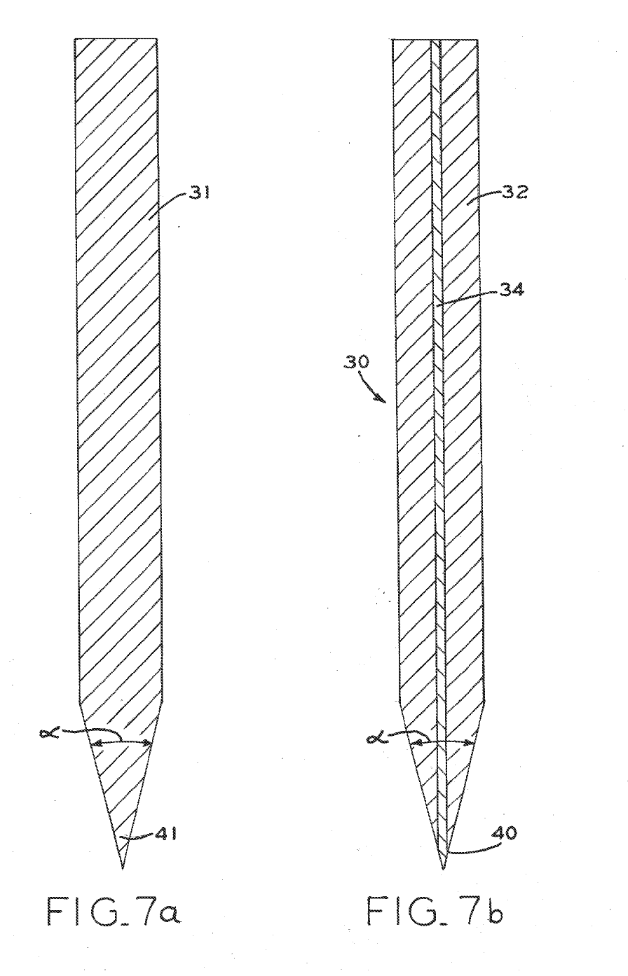

Turning now to FIG. 7a, monolithic wire 31 may terminate in pointed tip 41, such as by having a steadily reducing diameter to define a generally conical point. Pointed tip 41 may be appropriate for certain applications, such as a neural electrode, by aiding insertion into the implant site. Specifically, by adjusting angle α located at pointed tip 41, the sharpness of wire 31 can be increased or decreased to adjust the amount and nature of the forces experience by the distal end of wire 31 during penetration into tissue. In one exemplary embodiment, angle α may be as small as 5°, 10°, or 15° and as large as 20°, 25°, or 30°, or may be any angle within any range defined by any two of the foregoing values.

### Bimetallic Wire

1. Material Characteristics

Referring now to FIGS. 2a and 2b, bimetallic wire 30 is an elongate wire product constructed of two discrete materials each having a circular cross-section. Outer shell 32 is formed as a sacrificial sheath or tube made of a first biodegradable material, while core 34 is formed as an implantable, permanent or semi-permanent electrode made of a non-biodegradable material. In an exemplary embodiment, outer shell 32 is formed as a uniform and continuous surface or jacket such as a tube with a generally annular cross-sectional shape, and core 34 completely fills shell 32 in cross-section such that shell 32 and core 34 cooperate to form a solid cross-section. Wire 30 is thus formed as a wire product which may be coiled, braided, or stranded as desired, as well as spooled for transport, storage and dispensation.

Exemplary metals used in outer shell 32 are ferromagnetic, such that bimetallic wire 30 is compatible with high-speed magnetic insertion. Further, shell 32 is formed from a bioabsorbable material or alloy, such that shell 32 can be allowed to degrade inside the body over time, eventually leaving only the exposed core 34. Finally, shell 32 has sufficient strength and stiffness to support bimetallic wire 30 during penetration into the implantation site (e.g., the brain or another in vivo site) while avoiding bending or buckling of bimetallic wire 30 during such implantation.

In one exemplary embodiment, outer shell 32 is made of Fe or an Fe-alloy. Fe, an essential component of a variety of bodily enzymes, degrades rapidly inside the human body. Additional details of Fe-alloy materials useable for shell 32 are described in U.S. Patent Application Publication No. 2011/0319978, filed Jun. 24, 2011 and entitled BIODEGRADABLE COMPOSITE WIRE FOR MEDICAL DEVICES (Attorney Docket FWM0137-03), the entire disclosure of which is hereby expressly incorporated by reference herein. Other suitable iron alloys include Fe(II) and Fe(III).

In another exemplary embodiment, shell 32 of bimetallic wire 30 is an iron-manganese alloy (Fe—Mn) including an additional constituent element (X) which protects against pitting corrosion. This additional element (X) may include chromium (Cr), molybdenum (Mo), nitrogen (N) or any combination thereof. Exemplary such alloys are disclosed in international patent application Serial No. PCT/US2013/049970, filed Jul. 10, 2013 and entitled BIODEGRADABLE ALLOY WIRE FOR MEDICAL DEVICES (Attorney Docket FWM0166-01), which is assigned to the assignee of the present application, the entire disclosure of which is hereby expressly incorporated herein by reference.

Other suitable biodegradable materials for shell 32 include Mg and Mg-based alloys such as magnesium ZM21 and Mg(II).

Optionally, shell 32 may be partially or fully coated with a biodegradable polymer 35 (FIG. 2b) that may be drug-eluting. Suitable biodegradable polymers include poly-L lactic acid (PLLA) and poly-L glycolic acid (PLGA), for example.

Candidate metals for core 34 include materials which are biocompatible and suitable to transmit electrical signal. In one exemplary embodiment, core 34 is formed of Pt (or, in the alternative, a Pt-alloy, such as Pt—Ir), which is biocompatible and an exemplary conductor of electricity, and which may be used as a semi-permanent or permanent implant. In another embodiment, Au and Au alloys may also be used to form core 34. In yet another embodiment, W or W alloys may be used for core 34.

2. Geometric Characteristics

Referring to FIGS. 2a and 2b, bimetallic wire 30 is sized, shaped and constructed so that it may be magnetically driven into an implant site (e.g., a brain) and subsequently employed to convey electricity to or from the implant site throughout a long service life.

As noted above, bimetallic wire 30 is arranged such that shell 32 and core 34 are initially implanted as a single unit, with shell 32 providing mechanical strength for insertion of wire 30, but then slowly degrading in vivo to leave only the exposed core 34. Core 34, on the other hand, need not provide significant material strength for the initial insertion, but is constructed to function as an electrode after the implantation process is complete. Thus, the diameters D_{B} and D_{C} of shell 32 and core 34 respectively (FIG. 2a) can be independently set according to the design goals of each.

In one exemplary embodiment, outer diameter D_{B} of bimetallic electrode 30 may be as small as 13 μm, 25 μm, or 75 μm, or as large as 100 μm, 150 μm, or 200 μm, or may be any size within any range defined by any two of the foregoing values. Also, as previously described with respect to diameter D_{M} of monolithic wire 31, sizing the diameter D_{C} of core 34 at less than or equal to, for example, the diameter of a human hair, prevents or minimizes attack of the permanent or semi-permanent core 34 by the brain's natural physical defenses. Accordingly, diameter D_{C} is between 12 μm and 25 μm for an exemplary bimetallic wire 30 used as a neural electrode.

In addition to material selection, the other characteristics of bimetallic wire 30 may determine its biodegradation rate. For example, the thicknesses of shell 32 of bimetallic wire 30 may be selected to control the biodegradation rate of shell 32, with relatively thicker constructs requiring more time for biodegradation, and relatively thinner constructs requiring less time for biodegradation. Further, the geometry of shell 32 and/or the overall formed device may result in certain regions of bimetallic wire 30 being exposed to body tissue to a greater extent than other regions of bimetallic wire 30, which may affect the biodegradation rate. When outer shell 32 is formed of a more rapidly degrading material the degradation process is expected to consume outer shell 32 and leave continuous core 34 at a correspondingly rapid pace.

Although round cross-sectional wire forms are shown in the Figures of the present application and described further below, non-round wire forms may also be produced using the materials disclosed herein. Exemplary non-round forms include polygonal cross-sectional shapes such as rectangular cross-sectional shapes, which may be used for shell 32, core 34 or both.

Turning now to FIG. 7b, bimetallic wire 30 may terminate in pointed tip 40, such as by having a steadily reducing diameter to define a generally conical point. Pointed tip 40 may be appropriate for certain applications, such as a neural electrode, by aiding insertion into the implant site. As described above with respect to monolithic wire 31, angle α may be adjusted to adjust the sharpness of wire 30 and thereby influence implantation characteristics. In one exemplary embodiment, angle α may be as small as 5°, 10°, or 15° and as large as 20°, 25°, or 30°, or may be any angle within any range defined by any two of the foregoing values.

Further detail regarding the construction and geometry of a bimetallic wire in accordance with the present disclosure can be found in U.S. Pat. Nos. 7,420,124, 7,501,579 and 7,745,732, filed Sep. 13, 2004, Aug. 15, 2005 and Jan. 29, 2009 respectively and all entitled DRAWN STRAND FILLED TUBING WIRE, the entire disclosures of which are hereby expressly incorporated herein by reference.

### Wire Production

An alloy in accordance with the present disclosure is first formed in bulk, such by casting an ingot, continuous casting, or extrusion of the desired material. This bulk material is then formed into a suitable pre-form material (e.g., a rod, plate or hollow tube) by hot-working the bulk material into the desired pre-form size and shape. For purposes of the present disclosure, hot working is accomplished by heating the material to an elevated temperature above room temperature and performing desired shaping and forming operations while the material is maintained at the elevated temperature. The resulting pre-form material, such an ingot, is then further processed into a final form, such as a rod, wire, tube, sheet or plate product by repetitive cold-forming and annealing cycles. In one exemplary embodiment, this further processing is used to fabricate wires 30 and/or 31, as further described below.

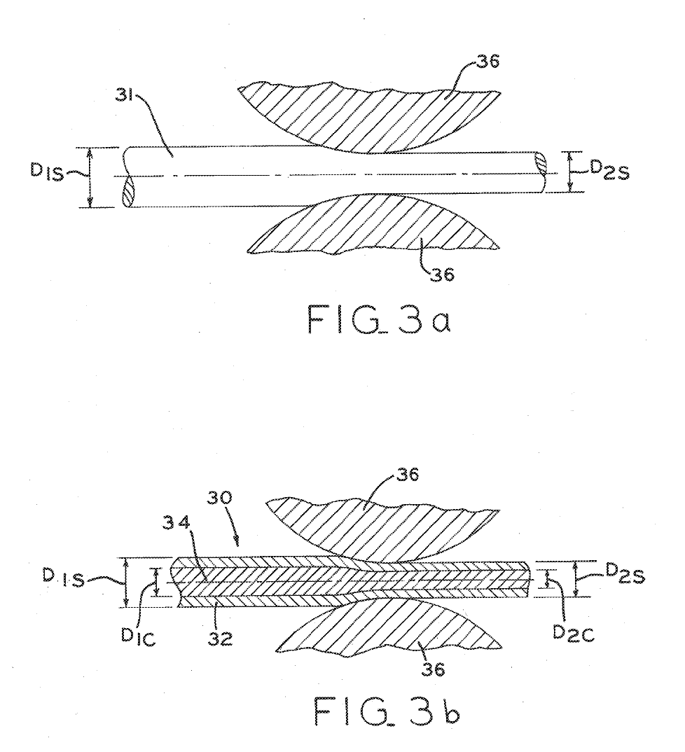

Monolithic wire 31 may be initially produced using conventional methods, including a schedule of drawing and annealing in order to convert the pre-form material (such as an ingot or rod) into a wire of a desired diameter prior to final processing. That is, the pre-form material is drawn through a die 36 (FIG. 3a) to reduce the outer diameter of the ingot slightly while also elongating the material, after which the material is annealed to relieve the internal stresses (i.e., retained cold work) imparted to the material by the drawing process. This annealed material is then drawn through a new die 36 with a smaller finish diameter to further reduce the diameter of the material, and to further elongate the material. Further annealing and drawing of the material is iteratively repeated until the material is formed into a wire construct ready for final processing into monolithic electrode 31.

To form bimetallic wire 30 (FIG. 2a), core 34 is inserted within shell 32 to form an initial wire construct, and an end of the wire construct is then tapered to facilitate placement of the end into a drawing die 36 (FIG. 3b). The end protruding through the drawing die 36 is then gripped and pulled through the die 36 to reduce the diameter of the construct and bring the inner surface of shell 32 into firm physical contact with the outer surface of core 34. More particularly, the initial drawing process reduces the inner diameter of shell 32, such that shell 32 closes upon the outer diameter of core 34 such that the inner diameter of shell 32 will equal the outer diameter of core 34 whereby, when viewed in section, the inner core 34 will completely fill the outer shell 32 as shown in FIG. 2a.

The step of drawing subjects wire 30 or 31 to cold work. For purposes of the present disclosure, cold-working methods effect material deformation at or near room temperature, e.g. 20-30° C. In the case of bimetallic electrode 30, drawing imparts cold work to the material of both shell 32 and core 34, with concomitant reduction in the cross-sectional area of both materials. The total cold work imparted to wire 30 or 31 during a drawing step can be characterized by the following formula (I):

$$cw = 1 - {( \frac{{D}_{2 S}}{{D}_{1 S}} )}^{2} \times 100 \% \qquad ( I )$$

wherein “cw” is cold work defined by reduction of the original material area, “D_{2S}” is the outer cross-sectional diameter of the wire after the draw or draws, and “D_{1S}” is the outer cross-sectional diameter of the wire prior to the same draw or draws.

Referring to FIGS. 3a and 3b, the cold work step may be performed by the illustrated drawing process. As shown, wire 30 or 31 is drawn through a lubricated die 36 having an output diameter D_{2S}, which is less than diameter D_{1S} of wire 30 or 31 prior to the drawing step. The outer diameter of wire 30 or 31 is accordingly reduced from pre-drawing diameter D_{1S} to drawn diameter D_{2S}, thereby imparting cold work cw. Drawn diameter D_{2S} may correspond to diameters D_{M} and D_{B} shown in FIGS. 1 and 2a and described above with respect to wires 30 and 31 respectively

Alternatively, net cold work may be accumulated in wire 30 or 31 by other processes such as cold-swaging, rolling the wire (e.g., into a flat ribbon or into other shapes), extrusion, bending, flowforming, or pilgering. Cold work may also be imparted by any combination of techniques including the techniques described here, for example, cold-swaging followed by drawing through a lubricated die finished by cold rolling into a ribbon or sheet form or other shaped wire forms.

In one exemplary embodiment, the cold work step by which the diameter of bimetallic wire 30 is reduced from D_{1S} to D_{2S} is performed in a single draw and, in another embodiment, the cold work step by which the diameter of bimetallic wire 30 is reduced from D_{1S} to D_{2S} is performed in multiple draws which are performed sequentially without any annealing step therebetween. For processes where drawing process is repeated without an intervening anneal on composite bimetallic wire 30, each subsequent drawing step further reduces the cross section of bimetallic wire 30 proportionately, such that the ratio of the sectional area of shell 32 and core 34 to the overall sectional area of bimetallic wire 30 is nominally preserved as the overall sectional area of bimetallic wire 30 is reduced. Referring to FIG. 3b, the ratio of pre-drawing core outer diameter D_{1c} to pre-drawing shell outer diameter D_{1S} is the same as the corresponding ratio post-drawing. Stated another way, D_{1C}/D_{1S}=D_{2C}/D_{2S}.

Thermal stress relieving, otherwise known in the art as annealing, at a nominal temperature not exceeding the melting point of either the first or second materials, is used to improve the ductility of the fully dense composite between drawing steps, thereby allowing further plastic deformation by subsequent drawing steps. Further details regarding wire drawing and annealing are discussed in U.S. Pat. No. 7,989,703, filed Feb. 27, 2009, entitled “Alternating Core Composite Wire,” assigned to the assignee of the present invention, the entire disclosure of which is incorporated by reference herein. When calculating cold work cw using formula (I) above, it is assumed that no anneal has been performed subsequent to the process of imparting cold work to the material. Heating wire 30 to a temperature sufficient to cause recrystallization of grains eliminates accumulated cold work, effectively resetting cold work cw to zero.

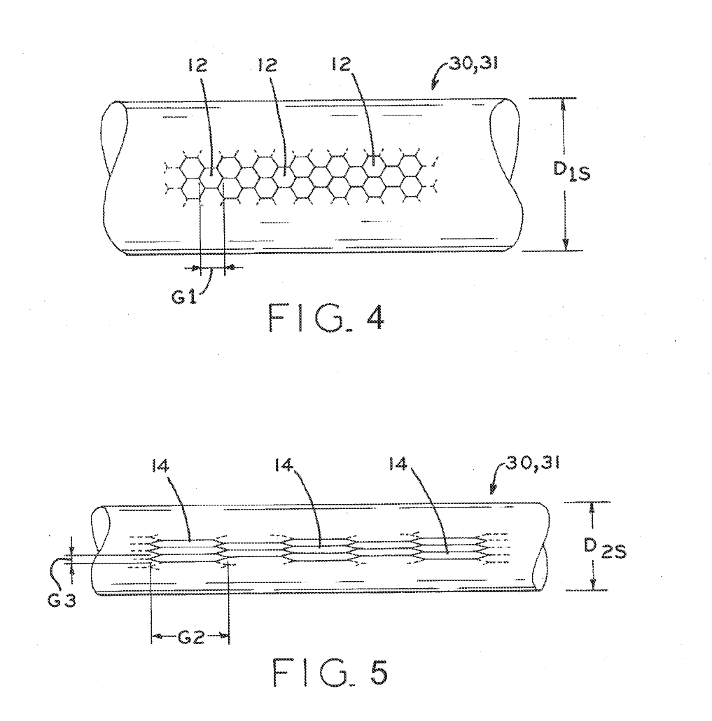

On the other hand, wire 30 or 31, subject to drawing or other mechanical processing without a subsequent annealing process, retains an amount of cold work. The amount of retained work depends upon the overall reduction in diameter from D_{1S} to D_{2S}, and may be quantified on the basis of individual grain deformation within the material as a result of the cold work imparted. Referring to FIG. 4, monolithic wire 31 is shown in a post-annealing state, with grains 12 shown substantially equiaxed, i.e., grains 12 define generally spheroid shapes in which a measurement of the overall length G1 of grain 12 is the same regardless of the direction of measurement. After drawing wire 31 (as described above), equiaxed grains 12 are converted into elongated grains 14 (FIG. 5), such that grains 14 are longitudinal structures defining an elongated grain length G2 (i.e., the longest dimension defined by grain 14) and a grain width G3 (i.e., the shortest dimension defined by grain 14). The elongation of grains 14 results from the cold working process, with the longitudinal axis of grains 14 generally aligned with the direction of drawing, as illustrated in FIG. 5.

The retained cold work of wire 31 after drawing can be expressed as the ratio of the elongated grain length G2 to the width G3, such that a larger ratio implies a grain which has been “stretched” farther and therefore implies a greater amount of retained cold work. By contrast, annealing wire 31 after an intermediate drawing process recrystallizes the material, converting elongated grains 14 back to equiaxed grains 12 and “resetting” the retained cold work ratio to 1:1.

Some exemplary biodegradable wire materials may have retained cold work in their final forms. For example, iron with as little as 50% retained cold work and as much as 90% or 99% retained cold work may be used for shell 32 where a high level of strength is desired.

Annealing processes may also be employed in wires with retained cold work, in which the annealing temperatures and/or durations are kept low enough to “soften” the materials while also preventing recrystallization of the material. For bimetallic wire 30, the softening point of the constituent materials is controlled by introducing cold work into the composite structure after joining the metals as described above. For either monolithic wire 31 or composite wire 30, deformation energy is stored in the cold-worked structure, and this energy serves to reduce the amount of thermal energy required for stress relief of the wire material. This processing facilitates annealing of the composite structure at temperatures in the range of 40% to 50% of the melting point of the material, such that a low-temperature annealing process provides ductility to the metal wire material without converting elongated grains 14 back to equiaxed grains 12. Such ductility facilitates spooling of the wire, as discussed below, and renders the wire suitable for in vivo uses where low ductility would be undesirable.

Upon completion of production of wires 30, 31 by drawing, cold work and/or annealing as described above, a continuous wire may be produced having a length of, e.g., at least 10, 20 or 30 meters, and as much as 100, 200 or 400 meters, or any length in any range defined by any of the foregoing values. This length of wire may be wound onto a spool for, e.g., transport, storage and dispensation.

### Helically Threaded Electrodes

After a wire, such as wire 30 or 31, has been inserted into an implant site at the desired depth and location, additional axial (i.e., depth) adjustment of the wire at the implant site may be desired. For example, wire 30 or 31 may be initially magnetically inserted using the system described above (or a wire may be inserted by any suitable alternative method). After this initial insertion, it may be desirable to slightly move the wire axially into or out of the implant site to precisely position the tip of the electrode at a particular implant depth. In a brain application, for example, such fine depth adjustment may facilitate access to a particular desired neural region.

As described in further detail below, such fine axial adjustments may be effected by incorporation of a helical groove or threadform in the outer surface of wire 30 or 31. As described in detail below, such helical grooves may be formed by various methods to create threaded wires 68 or 82, which may be identical to wires 30 or 31 except for the helical threadform on the outer surface of the wire (or, where wires 68 or 82 are bimetallic wires as shown, on the outer surface of the shell of the wire construct).

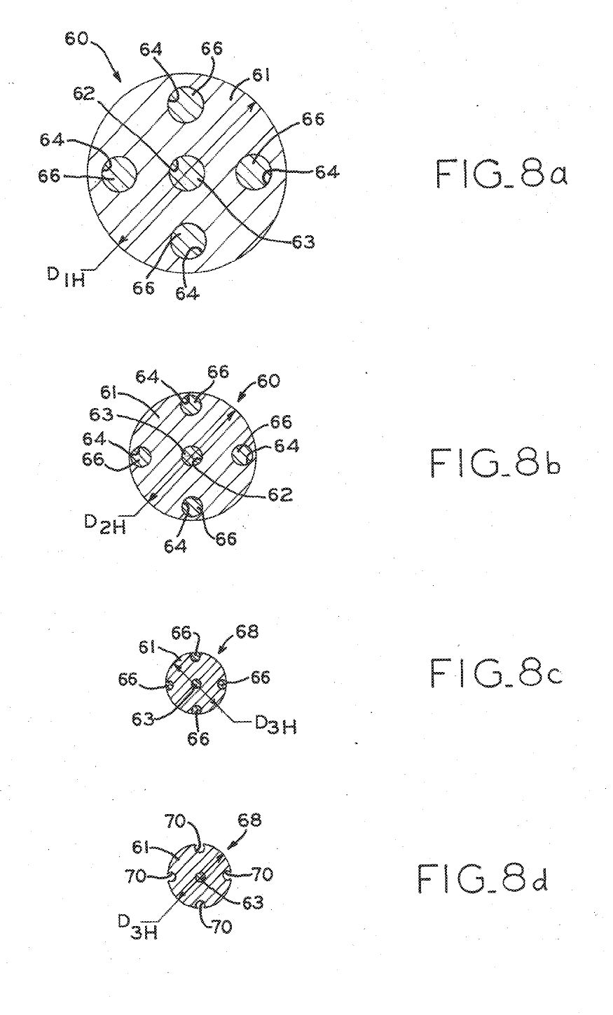

Such helical threads act as a “screw thread” when one of wires 68 or 82 is inserted into the implant site, engaging surrounding bone and other tissue to convert rotation of wire 68 or 82 into axial movement. In order to control the amount of axial movement of wire 68 or 82 for a given amount of rotation, the pitch of the helical thread point may be varied for a particular application. For purposes of the present disclosure, thread pitch may be expressed as a function of final wire diameter D_{3H} (FIGS. 8d and 11 e). Generally speaking, it is contemplated that the threadform pitch may range from very fine, i.e., a pitch equal to ½ of the finished wire diameter D_{3H} (i.e., 0.5*D_{3H}) to very coarse, i.e., about 100 times the finished wire diameter D_{3H}. In some exemplary embodiments, the pitch may be as small as (0.5*D_{3H}), (1*D_{3H}), or (2*D_{3H}), or as large as (50*D_{3H}), (75*D_{3H}), or (100*D_{3H}), or may be any value within any range defined by the foregoing values.

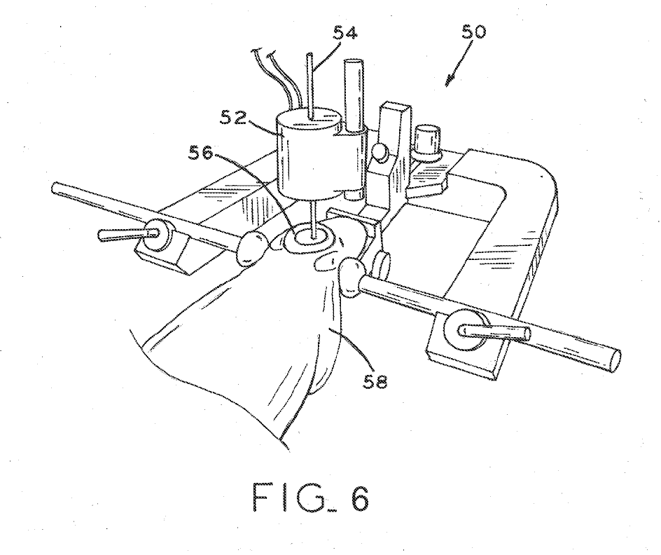

In use, wire 68 or 82 may first be magnetically inserted to an approximate implant depth by insertion system 50, shown in FIG. 6 and described in further detail below. With wire 68 or 82 thus inserted, torsion may be applied to wire 68 or 82 from outside the implant site, preferably near the tissue to maximize torque transmission. This torsion rotates wire 68 or 82 about its longitudinal axis. As this rotation occurs, bone and/or other tissue engaged with the helical threadforms 72 or 77 causes axial translation of wire 68 or 82 in the manner of a screw driving into a substrate material. In this way, rotating wire 68 or 82 after initial insertion at the implant site raises or lowers wire 68 or 82 such that the depth of insertion can be modified after initial implantation with a high degree of precision.

Methods of producing wires 68, 82 with helical threadforms 72, 77 respectively are described in detail below.

1. Filament Based Production

In one embodiment shown in FIG. 10a, wire 68 including helical threading may be used as a magnetically insertable electrode wire. The helical threading of wire 68 is applied by forming apertures through a metal length, filling those apertures with a solid filament, repeatedly drawing the length into a fine wire having longitudinal cavities, and finally twisting the wire so that those cavities assume a helical pattern. Each of these steps is described in further detail below.

FIG. 8a shows a cross-section of a wire construct, in the form of rod 60, including parent material 61 having central aperture 62 and a plurality (e.g., four) of surrounding apertures 64 formed through parent material 61. In an exemplary embodiment, diameter D_{1H} of rod 60 may be as little as 0.5 inches, 0.75 inches or 1.0 inch, or may be as large as 1.5 inches, 2.0 inches, or 3.0 inches. It is also appreciated that larger diameters D_{M} may be used as required or desired for a particular application. For example, large-scale production of extremely long continuous lengths of wire 68 may be accomplished with a starting material of rod 60 having a diameter D_{1H} as large as 6 inches, 8 inches or 10 inches. Diameter D_{1H} may be any value within any range defined by any of the foregoing values.

Where wire 68 (made from rod 60) is used in a magnetic insertion device 50 (as described herein), parent material 61 of rod 60 may be made of any of the materials described above with respect to monolithic wire 31, such as Fe—Pt alloy metals as described in detail above. Where central aperture 62 is included, parent material 61 of rod 60 may be made of any of the materials suitable for use as shell 32 of bimetallic wire 30, such as Fe or Fe alloys, also described in detail above. However, it is also contemplated that wire 68 may be inserted by other (i.e., non-magnetic) systems and methods, e.g., through a delivery cannula. In such non-magnetic insertion applications, wire 68 may be made from non-ferromagnetic materials such as stainless steel, Co—Cr superalloys, and platinum alloys.

Referring still to FIG. 8a, apertures 64 are formed as nonconcentric voids extending the length of rod 60, and are aligned symmetrically around the longitudinal axis of rod 60. Apertures 64 may have any shape, alignment, or number necessary for a desired application, with four apertures 64 illustrated in four respective quadrants of parent material 61. Central aperture 62 is formed through the center of parent material 61 of rod 60, (i.e., central aperture is concentric with the longitudinal axis of rod 60. Central aperture 62 may be omitted, depending on whether the finished wire construct is designed to include or exclude a core material 63 (distinct from parent material 61) as described below. Apertures 64 and central aperture 62 may be formed using any suitable method, such as drilling, gun-drilling or electric discharge machining (EDM).

Once apertures 62, 64 are formed in rod 60, filaments 63, 66 are respectively inserted into apertures 62, 64 using known methods. Central filament 63 may be made of any of the materials suitable for use as core 34 of bimetallic wire 30, such as non-bioabsorbable metals as described in detail above. Peripheral filaments 66 may include various materials, such as materials amenable to removal from apertures 64 (e.g., by physical separation, melting, etc.) after the drawing of rod 60 into wire 68 is completed. In some embodiments, filaments 66 could be made of (1) platinum, (2) fatigue resistant alloys, such as ASTM F 562 or ASTM F 590, (3) sensitized stainless steel or a mild steel, (4) magnesium-based alloy, (5) silver, (6) nitinol (an alloy made of 50% nickel and 50% titanium), (7) Fe—Mn alloys such as Fe35Mn, or (8) any other suitable metallic material desired for the applicable situation.

The hardness of parent material 61 and filaments 66 can influence the deformation and migration of apertures 64 during the iterative drawing process. If filaments 66 are extremely hard metallic materials and parent material 61 is softer than filaments 66, the deformation of filaments 66 during drawing (or other processing) will be slight and the cross-sectional geometry of filaments 66 will not remain proportional to parent material 61. However, if filaments 66 are either 1) soft metallic materials relative to parent material 61 or 2) materials with similar properties to parent material 61, the deformation will be proportionate across the wire section and the overall cross-sectional geometry will be substantially preserved during processing.

Rod 60, with filaments 63 and/or 66 embedded therein, is then subjected to a drawing and thermal-treatment process by passing the assembled wire construct through die 36 as shown in FIGS. 3a and 3b. Details of the iterative drawing process to reduce the overall diameter D_{1H} to an intermediate smaller diameter D_{2H} and then a finished diameter D_{3H} are described above. Where central aperture 62 and central filament 63 are included, the drawing process relating to bimetallic wire 30 is utilized as shown in FIG. 3b. Where central aperture 62 and central filament 63 are omitted, the drawing process relating to monolithic wire 31 is utilized as shown in FIG. 3a.

The iterative drawing process is illustrated by comparison of in FIGS. 8a, 8b and 8c. As mentioned previously, the initial diameter D_{1H} of rod 60 may range from 0.5 inches to as much as 10 inches. A finished wire 68, however, is substantially less than 1 mm in diameter in order to be suitable for use as, e.g., a neural electrode. In some instances, wire 68 may have a finished diameter as small as 5 μm in diameter, as described above. In order to effect this large reduction in diameter, rod 60 is subjected to a cold-working process via repeated drawing. After a first draw, annealing or thermally-treating (intermittently) rod 60 may be performed to rendering rod 60 more ductile (e.g., by allowing grain regrowth) and thereby facilitate continued drawing. Rod 60 is then drawn again to further reduce the diameter, from D_{2H} to D_{3H}. For illustration, FIGS. 8a-8c show only two draws used to create wire 68 from rod 60, though it is appreciated that any number of draw/anneal iterations may be utilized depending on material properties, starting and finishing sizes, etc.

Further detail regarding the production of wire 68 in accordance with the present disclosure can be found in U.S. Pat. No. 7,020,947 filed Sep. 23, 2003 and entitled METAL WIRE WITH FILAMENTS FOR BIOMEDICAL APPLICATIONS, and in U.S. Pat. No. 7,490,396, filed Jan. 24, 2006 and entitled METHOD OF MAKING METAL WIRE WITH FILAMENTS FOR BIOMEDICAL APPLICATIONS, the entire disclosures of which are hereby expressly incorporated herein by reference.

Referring to FIG. 8c, after a final diameter D_{3H} of wire 68 has been achieved, filaments 66 still remain deposited within apertures 64. As illustrated, filaments 66 migrated from an internal position, in which filaments were completely surrounded in cross-section by parent material 61, to their final position in which filaments 66 are exposed at the outer periphery of wire 68. This exposure facilitates removal of filaments 66 by a number of different processes. For example, a section of wire 68 may be placed into an acid bath that would attack the embedded filaments 66, but would not affect parent material of wire 68, thereby leaving open cavities 70. Filaments 66 may also be removed from wire 68 via a biodegradable or thermally activated corrosive attack process, whereby filaments 66 are leached out to leave cavities 70 in wire 68. Filaments 66 may also be removed from wire 68 via a micro or nano machining process, whereby filaments 66 are mechanically removed to leave cavities 70 in wire 68.

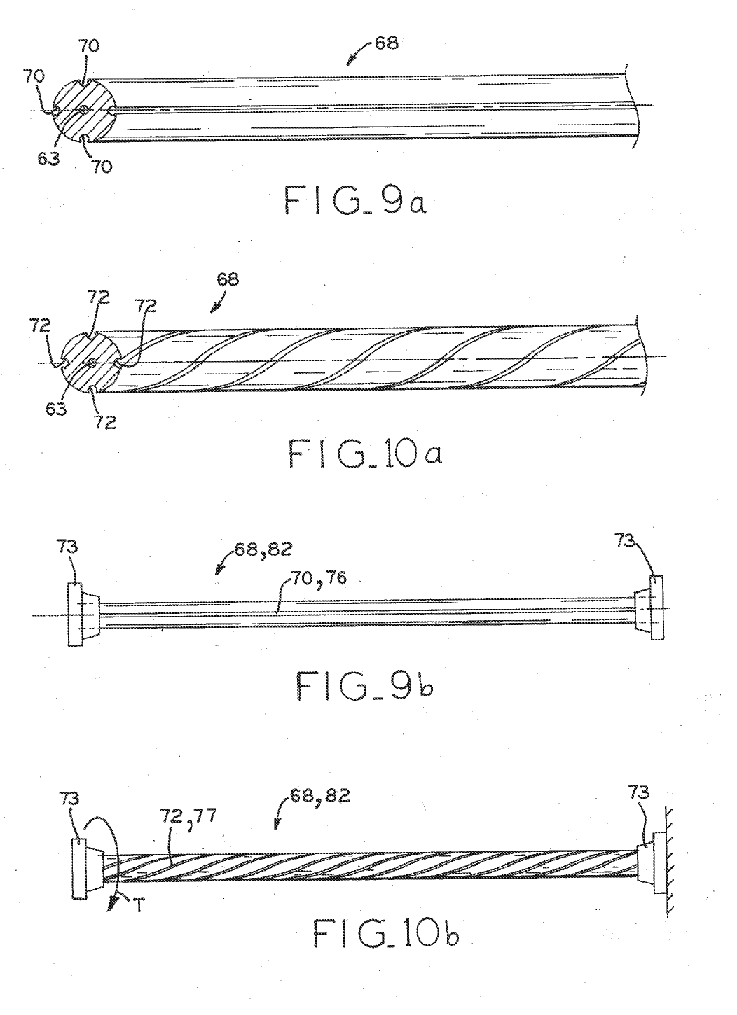

Turning now to FIGS. 9a and 9b, wire 68 is shown after the drawing process and the removal of filaments 66. As illustrated, wire 68 has four straight longitudinal cavities 70 extending along a path parallel to the longitudinal axis of wire 68. In order to form the final “screw thread” form of wire 68 (as shown in FIG. 10a), wire 68 is plastically deformed by twisting. For example, referring to 9b, the ends of wire 68 can be secured between grips 73 of a twisting machine, and one or both of grips 73 may be subsequently twisted (i.e., along direction T) to reform longitudinal cavities 70 into helical threads 72.

In an exemplary embodiment, the ambient temperature around wire 68 is elevated during this twisting process to alleviate build-up of internal stresses within parent material 61. In a further exemplary embodiment, such twisting is performed in a continuous production process, e.g., grips 73 are replaced by unwinding and winding spools that are rotated relative to one another along direction T to convert longitudinal cavities 70 into helical threads 72.

2. Jacketed Production

In an alternative production method, wire 82 made in accordance with the present disclosure includes helical threading applied by first cutting grooves along the outer surface of a length of parent material 71, jacketing that length of parent material 71 in a tubular jacket 79 made of, e.g., steel, and filling the space between jacket 79 and the parent material 71 with a buffer material 80 to create a wire drawing assembly 84. This assembly 84 is then subjected to one or more draws, such as an iterative draw/anneal cycle described in detail above, thereby converting the entire combination of jacket 79, parent material 71 and buffer material 80 into a fine wire construct. From this construct, an intermediate fine wire construct may be removed from jacket 79 and buffer material 80 for further processing into helical wire 82. Each of these steps is described in further detail below.

FIGS. 11a-11e show a progression of the production of wire 82 from rod 74. Rod 74 (FIG. 11a) may be initially procured as a smooth solid cylinder defining an initial diameter D_{1H}, which is the same initial diameter as rod 60 shown in FIG. 8a and described in detail above. In an exemplary embodiment, the parent material 71 of rod 74 is Fe or an Fe alloy, if rod 74 includes central aperture for eventual creation of a bimetallic implant wire construct similar to bimetallic wire 30. Parent material 71 of rod 74 may be, e.g., an Fe—Pt alloy for eventual creation of a monolithic implant wire construct similar to monolithic wire 31. Like rod 60 described above, any suitable alloy in accordance with the present disclosure may be used in conjunction with the present jacketed production method for screw-thread creation.

However, rod 60 has a plurality (e.g., six) of grooves 76 formed (e.g., by machining) in an outer periphery thereof, rather than peripheral apertures 64 of rod 60. Central aperture 75 is formed along the longitudinal axis of rod 74 (i.e., is coaxial with rod 74) and spans its axial length, similar to central aperture 62 of rod 60. Central aperture 75 may be formed by similar methods used for central aperture 62, e.g., drilling, gun-drilling or EDM. When central aperture 75 is formed, core material 78 is placed therein prior to the drawing process. Materials used for core material 78 may be the same as described above for core materials 34 and 63, e.g., platinum or another, electrically conductive and non-bioabsorbable metal.

After formation of grooves 76 and aperture 75, rod 74 is assembled with jacket 79 and buffer material 80 as shown in FIG. 11b in preparation for the drawing process. Rod 74 is placed into the cylindrical void within extrusion jacket 79, which is a tubular cylinder, such that an even and consistent buffer space between the outer surface of rod 74 and the inner surface of extrusion jacket 79 is formed. That is to say, rod 74 is made to be coaxial with jacket 79 such that the shape of the void created by the buffer space is a tubular cylinder. With rod 74 received and evenly spaced within jacket 79, buffer material 80 is used to fill the tubular void between the interior surface of jacket 79 and the exterior surface of rod 74, including each groove 76.

Extrusion jacket 79 and buffer material 80, like grooves 76 and central aperture 75, extend along the entire length of rod 74 so that during the subsequent drawing process (described below), the geometric proportion (i.e., cross-sectional geometry) of grooves 76 along the surface of rod 74 is substantially maintained. Buffer material 80 may be any flowable material which, when captured within the space between rod 74 and jacket 79, provides rigidity sufficient to evenly transfer pressure from jacket 79 to rod 74 during the drawing process without itself compressing or substantially deforming. Exemplary materials for buffer material 80 include 1) Fe, Fe-alloys or steel, 2) ceramic powder or boron nitride, or 3) other meltable polymers and/or flowable materials meeting the requirements listed above.

With drawing assembly 84 thus completed, drawing of the overall construct may begin. This drawing is done according to known methods, such as the method described above using die 36 for production of wires 30, 31 and 68. Further detail regarding exemplary drawing processes in accordance with the present disclosure can be found herein, and in U.S. Pat. No. 7,020,947 filed Sep. 23, 2003, and entitled METAL WIRE WITH FILAMENTS FOR BIOMEDICAL APPLICATIONS, the entire disclosure of which is hereby expressly incorporated herein by reference.

A first drawing process may reduce the overall diameter of assembly 84 from D_{1D} to D_{2D}, as illustrated by a comparison of FIGS. 11b and 11 c. As this reduction occurs, diameter D_{1H} of rod 74 is reduced by a proportional amount to diameter D_{2H}. That is, the ratio of reduction of rod 74 (i.e., D_{1H}/D_{2H}) is substantially equal to the ratio of reduction of the overall wire drawing assembly (i.e., D_{1D}/D_{2D}). In addition, the even pressure applied from die 36, through jacket 79 and then through buffer material 80 to parent material 71 and central filament 78 (if present) ensures that the overall cross-sectional geometry of rod 74 is precisely controlled and, in some cases, substantially preserved as the initial diameter D_{1D} is reduced. That is to say, buffer material 80 cooperates with jacket 79 to preserve the evenness and geometric proportionality of grooves 76 throughout the diameter reduction, by protecting rod 74 from frictional forces and uneven pressures that would otherwise be applied by directly drawing rod 74 through die 36. In this way, rod 74 maintains its desired shape and geometry through the drawing process.

Referring from FIG. 11c to FIG. 11d, an additional drawing process may be performed to further reduce diameters D_{2H}, D_{2D} to diameters D_{3H}, D_{3D}. At this point, wire 82 is formed and contained within jacket 79. Additional iterative drawing processes may be used, as described in detail above with respect to rod 60 and the resulting wire 68.

Extrusion jacket 79 and buffer material 80 are then removed from wire 82 by similar methods used for removing filaments 66 from wire 68. Specifically, buffer material 80 is removed from jacket 79, at which point wire 82 can be withdrawn from jacket 79. In one embodiment, removal of buffer material 80 is effected by first using a chemical etch to remove the iron or steel material of jacket 79. Then, if buffer material 80 is metallic, etching will also be removed to expose grooves 76. If buffer material 80 is a powder (e.g., ceramic), mechanical cleaning, such as ultrasonic cleaning can be used to remove material 80 from grooves 76. In some cases, a subsequent light etch of the mechanically cleaned wire 68 may be used to remove any residual material in grooves 76.

In order to convert the previously-machined, exposed grooves 76 into the desired helical threadform 77 (FIG. 11e), wire 82 is twisted in similar fashion to wire 68 (described in detail above). For example, FIGS. 9b and 10b illustrate wire 82 captured by grips 73, which rotate relative to one another as described above. Application of heat and continuous processing methods may also be employed as described above.

The resulting finished wire 82, shown in FIG. 11e, has a finished diameter D_{3H} similar to that of wire 68 made through filament based production described above with respect to FIGS. 8a-8d.

In one exemplary embodiment, magnetically inserted electrodes may be fabricated in the form of a spooled monolithic wire 31 having a diameter of, e.g., 25 microns. This exemplary wire may be formed from an ingot of Pt-40 wt. % Fe alloy, triple arc melted in high purity argon then drop cast into a cold copper mold to form the ingot. One exemplary ingot may measure 12.7 mm×150 mm, which may be homogenized at 1523 K for 2 hours, water quenched, and drawn by successive diamond die drawing as described in detail above to achieve a highly polished surface and a final diameter of 25 microns for wire 31.

Electrodes may be cut from the spool of wire 31 at a length of about 2 cm, and several of these electrodes can then loaded together into a custom-made clamp for parylene coating. Electrodes may then be coated in 2 microns thickness of parylene along half of each electrode length (the clamp held onto part of each electrode and thus prevented coating thereon) and removed from the clamp. Electrode tips can be cut with a scalpel leaving 7 mm of the electrode coated in parylene, from the cut tip to the point where the clamp held the electrode. The other end of each electrode can also be cut so that 3 mm of the electrode remained uncoated in parylene. Thus, each electrode was 10 mm in length, with 7 mm insulated in parylene and 3 mm with no insulation, and with the recording surface located at the end of the insulated portion of the electrode.

Referring to FIG. 6, a high-speed magnetic insertion instrument for inserting one of wires 30, 31, 68, 82 into brain tissue 56 of rat 58 is illustrated. In this exemplary embodiment, the high-speed magnetic insertion system 50 uses an induced magnetic field to accelerate the above-described monolithic wire 31 formed of Fe—Pt alloy into brain tissue. High voltage capacitors may be charged up to approximately 370 V so that they discharge current through coil 52 of insulated copper wire to induce a uniform magnetic field within the coil. Running glass capillary tube 54 through the coil can hold the wire (not shown) to insertion and guide the electrode during insertion. A small piece of refrigerator magnet is sufficient to hold the wire slightly above the coil prior to insertion. By discharging the capacitors, the magnetic field produced by coil 52 will accelerate the wire into coil 52, through glass capillary tube 54, and into brain tissue 56.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. An elongate, flexible wire comprising:
a maximum diameter between 5 μm and 200 μm; and
a composition comprising a combination of a ferromagnetic metal and a biocompatible metal.

2. The wire of claim 1, wherein said ferromagnetic metal contains at least one of iron, nickel, and cobalt.

3. The wire of claim 1, wherein said biocompatible metal contains at least one of gold, platinum, and tungsten.

4. The wire of claim 1, wherein said composition comprises between 20 wt. % and 70 wt. % iron and balance platinum.

5. The wire of claim 1, wherein the wire is a substantially cylindrical, monolithic construct having a diameter between 12 μm and 25 μm.

6. The wire of claim 1, wherein said maximum diameter narrows at an end of the wire such that the wire defines a pointed tip.

7. The wire of claim 1, wherein said pointed tip defines an angle between 5 degrees and 30 degrees.

8. The wire of claim 1, further comprising a helical threadform formed on an outer surface of said wire.

9. A bimetallic wire, made for insertion at an implant site, the wire comprising:
a shell made of a ferromagnetic metal;
a core disposed within said shell, said core made of a biocompatible metal, said core defining a core diameter between 5 μm and 25 μm.

10. The wire of claim 9, wherein said shell has an outer shell diameter between 75 μm and 200 μm.

11. The wire of claim 9, wherein said ferromagnetic metal contains at least one of iron, nickel, and cobalt.

12. The wire of claim 9, wherein said biocompatible metal contains at least one of gold, platinum, and tungsten.

13. The wire of claim 9, wherein an overall diameter of said wire narrows at an axial end of the wire such that the wire is pointed.

14. The wire of claim 9, wherein said bimetallic wire further comprises a helical threadform formed on an outer surface of said shell.

15. The wire of claim 9, wherein said core diameter is substantially equal to an inner diameter of said shell, such that said core completely fills said shell as viewed in cross-section.

16. An elongate, flexible electrode wire for use at an implant site, the wire comprising:
a maximum diameter between 5 μm and 200 μm;
a composition comprising a biocompatible metal; and
a helically-grooved outer surface.

17. The wire of claim 16, wherein said wire is a bimetallic wire comprising a shell and a core disposed within said shell, said shell having an outside shell diameter between 75 μm and 200 μm and an inside shell diameter substantially equal to an outside core diameter, such that said core completely fills said shell as viewed in cross-section.

18. The wire of claim 16, wherein said composition comprises a ferromagnetic metal containing at least one of iron, nickel, and cobalt.

19. The wire of claim 16, wherein said biocompatible metal contains at least one of gold, platinum, and tungsten.

20. The wire of claim 16, wherein an overall cross-sectional wire extent narrows at an end of the wire such that the wire is pointed.
